(19) United States
(12) Patent Application Publication
(10) Pub. No.: US 2010/0261219 A1
(43) Pub. Date: Oct. 14, 2010

(54) TEST METHOD OF BIOAVAILABILITY AND BIOEQUIVALENCE FOR XENOBIOTICS USING GENETIC PROFILING

(75) Inventors: Dong-Chool Kim, Daejeon (KR); Rodney J.Y. Ho, Mercer Island, WA (US)

Correspondence Address: KNOBBE MARTENS OLSON & BEAR LLP, 2040 MAIN STREET, FOURTEENTH FLOOR, IRVINE, CA, 92614, US

(73) Assignee: IBIOPHARM CO., LTD., SEOUL (KR)

(21) Appl. No.: 12/594,194

(22) PCT Filed: Jan. 7, 2009
(86) PCT No.: PCT/KR09/00077

§ 371 Date: Sep. 30, 2009

(30) Foreign Application Priority Data
Jan. 7, 2008 (KR) 10-2008-0001746
Jan. 6, 2009 (KR) 10-2009-0000948

(51) Int. Cl. C12Q 1/02

(52) U.S. Cl. 435/29

(57) ABSTRACT

Disclosed is a test method of bioavailability or bioequivalence for xenobiotics, comprising selection of test subjects (human or animal) based on the genetic information for metabolic enzymes or transporters that influence pharmacodynamics or pharmacokinetics for xenobiotics, and testing bioavailability or bioequivalence of the same.

Consideration of this method for applying genetic profiling information to improve analysis of a result from the bioavailability or bioequivalence test after the clinical test is also provided.

This application claims priority to Korean Patent Application Nos. 10-2008-0001746, 10-2009-0000948 filed on 7 Jan. 2008, 06 Jan. 2009, in the Korean Intellectual Property Office; the entire contents of which are hereby incorporated by reference.

### BACKGROUND OF THE INVENTION

1. Field of the Invention

The present invention relates to a test method of bioavailability or bioequivalence for xenobiotics, and more particularly, to a test method of bioavailability and bioequivalence of xenobiotics, including: (a) selection of a test subject (animal or human body) based on the genetic profiling information of metabolic enzymes or transporters that have influence on pharmacokinetics (PK) or pharmacodynamics (PD) for xenobiotics, (b) testing bioavailability or bioequivalence of the same, and (c) analysis of test results for bioavailability or bioequivalence of xenobiotics utilizing genetic profiling information of the subject after the test.

2. Description of the Related Art

Bioavailability (hereinafter abbrev. to “BA”) means the rate and extent of active ingredients or available substances in medicaments which reach action sites in the body of a subject.^{1,2}

Bioequivalence (hereinafter abbrev. to “BE”) means no statistically significant difference between any two kinds of formulations under comparison in terms of rate and extent of active ingredients or available substances in medicaments which reach action sites in the body of a subject, when these formulations were administered to the subject with the same doses and routes and methods of administration.^{1,2}

BA and BE are represented by PK parameters for AUC (area under blood concentration versus time curve) and for Cₘₐₓ (peak drug concentration in blood), both of which are indicators of the amount and rate of active ingredients or available substances in medicaments which are absorbed at action sites in the body of a subject respectively. If it is difficult to measure concentration of such ingredients or substances in blood, accumulated amount of urinary excretion of the ingredients or substances may be used as the indicator for absorption amount. Specifically, the BE test must satisfy strict statistical criteria to prove that a test formulation has the therapeutically equivalent effect to a control formulation.^{1,2}

The purpose of the BE test is to determine whether there is a difference between a test formulation and a control formulation of each dosage formulation. Therefore, if errors other than that contributed by the dosage formulations can be minimized, any difference between a test formulation and a control formulation in BE testing can be more effectively and efficiently determined.

Accordingly, the BE test is in general performed by the following 2×2 crossover study. Some participants for the test were divided into two treatment sequence groups in terms of standard two periods and two-sequence crossover design.

In a typical BE test, the participant of a first sequence is administered a control drug R during Period-1 while a test drug T is administered during Period-2. In contrast, for the participant of a second sequence, the test drug T is administered during Period-1 while the control drug R is administered during Period-2. A sufficient rest or drug free interval called a washout period between Period-1 and Period-2 may be required. Such test design refers to RT/TR design as shown in the following Table 1.

TABLE 1
RT/TR design

| Sequence | Period-1 | Washout | Period-2 |
|---|---|---|---|
| 1 | R | | T |
| 2 | T | | R |

The above crossover study may be an optimum study design for the BE test so as to eliminate a between-subject variability in residual variation. For this reason, the FDA′ and/or CPMP³ also recommend the BE test based on the crossover design. When a drug has an excessively long half-life, a parallel study design may be allowed. In this case, it should be noted that a principal hypothesis of the BE test is that drug clearance of the participant is not varied (that is, constant) throughout Period-1 and Period-2. However, such parallel study design is not substantially employed in practical cases since this study demands a great number of subjects, as compared to the crossover design.

According to BE guidances^{1,2,3,4} published by US FDA, EMEA and/or KFDA, when mean values of PK parameters (AUC, Cₘₐₓ) for a test drug and a control drug is logarithmically transformed and compared to each other, a difference of the transformed mean values of PK parameters between the test drug and the control drug, especially, 90% confidential interval of the difference, must be within the range of 80 to 125% in order to recognize bioequivalence. The confidential interval (often referred to as “CI”) may be calculated by Equation 1.

90% CI=EXP(Diff±t_{0.05,N-2}\sqrt{2σ_{w}²/N}) Equation 1:

wherein,

Diff: difference of logarithmically transformed mean values between the test drug and the control drug, σ_{w}²:residual variance (within-subject),

N: total number of participants in the BE study

From Equation 1, it can be understood that the range of CI in the BE test may vary proportionally to variability contributed within a subject (or among intra-subjects) and inverse proportional to the size of samples.

That is, for a drug having small within-subject variability, the CI is narrow as shown in Equation I and bioequivalence may be easily demonstrated (see FIG. 1).

However, some drugs having relatively large within-subject variability such as a highly variable drug (HVD) may have difficulty in reaching a desired confidential interval. FIG. 1 schematically illustrates the above results.

In general, HVDs are drugs exhibiting greater than 30% of within-subject or intra-subject variability for AUC and/or Cₘₐₓ as measured in BA/BE test.^{5,6}

Bioequivalence study reports filed with US FDA from 2003 to 2005, are analyzed and sorted according to HVDs in the following Table 2.⁷

TABLE 2
Number of BE studies of highly variable drugs
reviewed by the Division of Bioequivalence in
the Office of Generic Drugs from 2003 to 2005.⁷

BE studies

| Description | Number | % of total |
|---|---|---|
| RMSE of AUC and/or Cₘₐₓ ≧0.3 | 111 | 11 |
| RMSE of AUC and/or Cₘₐₓ ≦0.3 | 899 | 89 |
| Total number of drugs studied | 1010 | 100 |

RMSE: ANOVA root mean square error

About 11% of the reviewed BE studies corresponding to 111 of the BE studies were directed to HVDs. Since such lots of drugs are HVDs and a within-subject variability of each of the drugs is substantially more than 30%, they have a wider confidential interval. Thus, these drugs with high-intra-individual variability would be more difficult to remain within a range of 80 to 125% defined by a BE study guideline (see FIG. 1).

As for a drug having a large within-subject variability such as an HVD, larger participant numbers are required in order to prove the BE of the drug. From a standpoint of a pharmaceutical corporation who sponsors BE studies, it is very difficult to satisfy the above requirement in terms of costs and/or success rate. In addition, the drug studied must be administered to significantly larger number of participants, thus not being desirable in consideration of the subjects and protection of their human rights.

In this regard, the present invention describes a novel method employing genetic profiling information to reduce the number of subjects needed for BE studies, and to enhance the success rate of BA or BE tests for xenobiotics such as HVDs having large within-subject variability.

### SUMMARY OF THE INVENTION

Accordingly, the present invention is directed to solve problems of prior arts as described above and an object of the present invention is to provide a BA or BE test method for xenobiotics in a test subject using genomic profiling information of metabolic enzymes or transporters that have influence on pharmacokinetics (hereinafter abbrev. to “PK”) or pharmacodynamics (hereinafter abbrev. to “PD”) for xenobiotics.

Another objective of the present invention is to provide a BA or BE test method for xenobiotics in a test subject to design the test or to calculate the number of subjects required for the above test by analyzing genomic profiling information of metabolic enzymes or transporters that have influence on PK or PD for the xenobiotics.

Still another objective of the present invention is to provide a method of utilizing genetic profiling information from a test subject after the BA or BE test for xenobiotics.

In order to accomplish the above described objective, the present invention provides a BA or BE test method for xenobiotics in a test subject, including: screening genetic type (hereinafter referred to as “genotype”) of metabolic enzymes or transporters that have influence on PK or PD for xenobiotics to a test subject; identifying whether the screened genotype is wild type, heterozygous type or mutant type; and determining whether the test subject is included in the test or not, dependent upon the genotype.

According to the novel test method, if the genotype of the metabolic enzyme or transporter for xenobiotics is the heterozygous type, the heterozygous type may be included in test subjects where there is no significant difference between the heterozygous type and a wild type in terms of PK or PD thereof.

According to the current invention, if the genotype of the metabolic enzyme or transporter for xenobiotics is the heterozygous type, the heterozygous type may not be included in test subjects where there is a significant difference between the heterozygous type and a wild type in terms of PK or PD thereof.

According to the current invention, if the genotype of the metabolic enzyme or transporter for xenobiotics is the mutant type, the mutant type may not be included in test subjects.

The present invention also provides a BA or BE test method for xenobiotics in a test subject to design the test or to calculate the number of subjects required for the above test by analyzing genomic profiling information of metabolic enzymes or transporters that have influence on PK or PD for the xenobiotics.

The present invention also provides a method of utilizing genetic profiling information after the BA or BE test, comprising: profiling genotypes of test subjects if there is an outlier affecting a conclusion of the analysis for BA or BE test; classifying the genotype into a wild type, a heterozygous type or a mutant type and comparing PK or PD parameters per each genotype; and, if a significant difference of the PK or PD parameters is recognized between different genotypes, analyzing a result of the comparison except the outlier depending on the genotypes.

The present invention provides a BA or BE test method for xenobiotics to reduce side effects possibly observed in a test subject, by carrying out the BA or BE test in consideration of genotypes of metabolic enzymes or transporters that have influence on PK or PD for the xenobiotics.

The present invention also provides a BA or BE test method for xenobiotics to shorten a test period by selecting a test subject having a wild type metabolic enzyme or transporter that has influence on PK or PD for xenobiotics to carry out the test.

Additionally, the present invention provides a BA or BE test method for xenobiotics, performed by utilizing the fact that there is a positive proportional relation between a within-subject variability and a between-subject variability for PK parameters, so as to design a result of the BA test for xenobiotics by at least one selected from a group consisting of crossover study, parallel study and repeated study or to utilize at least one evaluation procedure selected from a group consisting of average BE, individual BE and population BE.

According to the foregoing inventive test method, the PK parameters may comprise at least one selected from AUC and Cₘₐₓ.

In order to reduce a between-subject variability of a xenobiotic having a biological half-life of more than 5 days, the present invention further provides a BA or BE test method for xenobiotics, comprising: selecting test subjects who have the same or similar genetic polymorphisms of metabolic enzymes or transporters that have influence on PK or PD for the xenobiotic, and carrying out a parallel BA or BE test for the test subject.

With regard to the BA or BE test method for xenobiotics according to the present invention, a test subject is selected in consideration of genetic properties of metabolic enzymes or transporters that have influence on PK or PD for xenobiotics in order to reduce the between-subject variability and the within-subject variability, so that the test method may increase statistical power, and more clearly identify differences between formulations to enhance success rate of the test.

According to this novel test method, it may be possible to decrease the number of test examples for the BE test (number of test participants or animals) so as to reduce the entire cost and time required for the test and, to reduce incidence of expressing side effects or harmful effects of the xenobiotics to the test subject thereby embodying improvement in view of protecting human rights of test subjects.

The present invention may also utilize genetic profiling information described above in analysis of results from the foregoing BA or BE test.

### BRIEF DESCRIPTION OF THE DRAWINGS

These and other objects, features, aspects, and advantages of the present invention will be more fully described in the following detailed description of preferred embodiments and examples, taken in conjunction with the accompanying drawings. In the drawings:

FIG. 1 is a schematic view illustrating 90% CI of a difference of mean values of log-transformed AUC or Cₘₐₓ between a test drug and a control drug in a BE test.

Referring to FIG. 1, the CI is proportional to an extent of a within-subject variability. Therefore, a highly variable drug (HVD) sometimes does not satisfy BE criteria even if the drug is substantially equivalent to the control drug in terms of the formulation aspect.

FIG. 2 is a graph illustrating correlation between a within-subject coefficient of variation (abbrev. to “CV_{w}”) and a between-subject coefficient of variation (abbrev. to “CV_{b}”), wherein CV_{w} and CV_{b}, were measured in relation to C. obtained in 50 BE tests according to the present invention and were expressed as natural log-transformed values thereof.

Referring to FIG. 2, the straight line indicates regressive line, the long dotted line exhibits 90% CI of the regressive line, and the short dotted line shows 90% CI of individual measurement points.

FIG. 3 is a graph illustrating the correlation between a CV_{w} measured in relation to Cₘₐₓ obtained in 50 BE tests according to the present invention and a 90% CI in relation to a difference of log-transformed mean Cₘₐₓ values between a test drug and a control drug, wherein CV_{w} and CI were expressed as natural log-transformed values thereof.

Referring to FIG. 3, the straight line indicates regressive line, the long dotted line exhibits 90% CI of the regressive line, and the short dotted line shows 90% CI of individual measurement points.

FIG. 4 is a graph illustrating correlation between a CV_{w} and a CV_{b}, wherein CV_{w} and CV_{b} were measured in relation to AUC obtained in 50 BE tests according to the present invention and were expressed as natural log-transformed values thereof.

Referring to FIG. 4, the straight line indicates regressive line, the long dotted line exhibits 90% CI of the regressive line, and the short dotted line shows 90% CI of individual measurement points.

FIG. 5 is a graph illustrating correlation between a CV_{w} measured in relation to AUC obtained in 50 BE tests according to the present invention and a 90% CI in relation to a difference of log-transformed mean Cₘₐₓ values between a test drug and a control drug, wherein CV_{w} and CI were expressed as natural log-transformed values thereof.

Referring to FIG. 5, the straight line indicates regressive line, the long dotted line exhibits 90% CI of the regressive line, and the short dotted line shows 90% CI of individual measurement points.

FIG. 6 is a graph illustrating variation in concentration of risperidone in blood plasma with time for seventeen (17) healthy adult participants, after oral administering a risperidone formulation of the present invention to all of the participants.

FIG. 7 is a graph illustrating concentration of risperidone in blood plasma for participants with wild type CYP2D6*10.

FIG. 8 is a graph illustrating concentration of risperidone in blood plasma for participants with heterozygous type CYP2D6*10.

FIG. 9 is a graph illustrating concentration of risperidone in blood plasma for participants with mutant type CYP2D6*10.

FIG. 10 is a flow chart illustrating selection of test subjects in BA/BE test of the present invention by utilizing genetic polymorphism information of metabolic enzymes or transporters that have influence on PK/PD.

FIG. 11 is a flow chart illustrating analysis of test results after BA/BE tests of the present invention by utilizing genetic polymorphism information of metabolic enzymes or transporters that have influence on PK/PD.

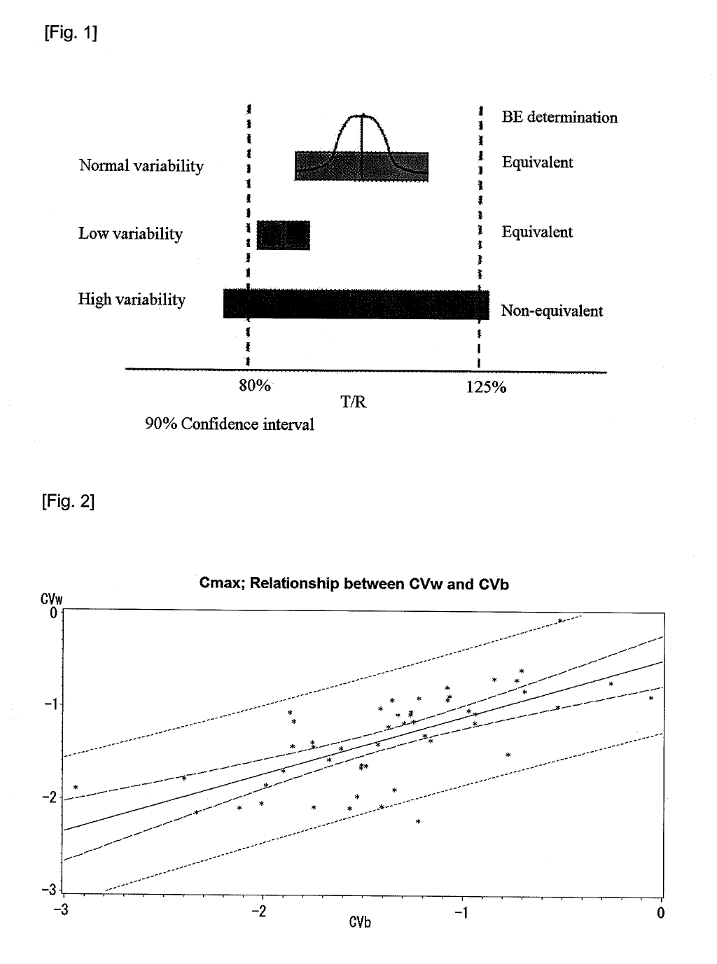

### DETAILED DESCRIPTION OF THE INVENTION

The BA or BE test method for xenobiotics according to the present invention may comprise: screening genotype of metabolic enzyme or transporter that has influence on PK or PD for xenobiotics to a test subject; identifying whether the genotype of metabolic enzyme or transporter is wild type, heterozygous type or mutant type; and determining whether the test subject is included or not, dependent on the genotype.

Xenobiotics used herein refer to medicaments applicable to human, veterinary medicines, bio medicines, and/or materials usable in manufacturing medicaments applicable to human or veterinary medicines.

Metabolic enzyme and transporter used herein refer to metabolic enzyme and transporter with effects on PK or PD for xenobiotics. For bio medicines, specific receptors often relate to PK/PD and this is also included in the concept of metabolic enzyme and transporter in a broad definition in the present invention.

As described above, when the genotype of metabolic enzyme or transporter is heterozygous type, the heterozygous type enzyme or transporter is preferably included in test subjects so far as the heterozygous type is not significantly different from the wild type for PK or PD.

Conversely, when the genotype of metabolic enzyme or transporter is the heterozygous type, the heterozygous type enzyme or transporter is preferably excluded from test subjects so far as the heterozygous type is significantly different from wild type for PK or PD. “Not significantly different from” means, for example, that the difference of mean values of Cₘₐₓ or AUC as PK parameters between for wild type and for heterozygous type is not noticeable, thus being around 5% or 1% of significance level as determined by statistical methods.

From the above description, if the genotype of the metabolic enzyme or transporter is mutant type, the mutant type is preferably excluded from test subjects.

In addition, the BA/BE test method of the present invention may include analysis of genetic profiling information of metabolic enzyme or transporter having influence on PK/PD for xenobiotics in the test subject to design a BA or BE test, or the calculation of the number of test examples required for the same test.

The present invention may also utilize the genetic profiling information of test subjects, after completing the BA or BE test, in order to determine whether a particular test subject must be included in the final analysis or not.

As for utilization of the genetic profiling information after the BA or BE test, the present invention may carry out profiling about the genotype of test subjects if there is an outlier affecting a conclusion of the analysis of BA or BE test.

After classifying the genotype of test subjects into wild type, heterozygous type and/or mutant type by profiling of genotypes of test subjects, if a significant difference of PK or PD parameters between individual genotypes is identified by comparing PK or PD parameters for the genotypes the results are preferably analyzed, except outliers dependent on the genotypes.

Furthermore, with regard to the BA or BE test method for xenobiotics according to the present invention, the test is performed by selecting the test subjects with only a wild type in consideration of genotypes of the metabolic enzyme or transporter that has influence on PK or PD for xenobiotics so as to reduce side effects or harmful effects possibly observed in test subjects, thereby embodying improvement in view of protection of the human rights of test subjects.

In accordance with the inventive BA or BE test method for xenobiotics, selecting a test subject with only a wild type metabolic enzyme or transporter that has influence on PK or PD for xenobiotics may shorten the period of examination.

For the test subjects with only wild type, concentration of xenobiotics in blood is rapidly reduced rather than the test subjects with mutant type. Therefore, the present invention may considerably reduce time taken in blood collection by selecting persons with only wild type as the test subjects.

It may also be expected that expression of the degree of side effects caused by a drug is decreased because of relatively lower concentrations of the drug in blood plasma.

Accordingly, the present invention reduces side effects possibly occurring during BA or BE test by selection of participants with wild type metabolic enzymes and transporters, thereby overcoming problems such as maintenance of participants and/or dropouts of the test.

The present invention describes a principal concept that selects test subjects (human or animal) based on the genetic profiling information of the metabolic enzyme or transporter that has influence on PK or PD for xenobiotics and performs BA or BE test for the test subjects. Thus, the present invention may utilize a variety of genetic profiling processes (such as RT-PCR, gene sequencing, gene chip, etc.) as well as BA or BE test methods conventionally used in the related art.

It is well known that genetic polymorphisms have influence on differences of PK or PD for individuals.

Preferred examples of genetic polymorphisms of cytochrome P450 metabolic enzymes are listed in Table 3, while preferred examples of genetic polymorphisms of phase-2 metabolic enzymes are listed in Table 4 as follows. In addition, Table 5 shows preferred examples of genetic polymorphisms of specific transporters.⁹

TABLE 3
Number of alleles with CYP polymorphisms
and functional effects thereof

| | Number | | |
|---|---|---|---|
| CYP | of | | Functional |
| gene | Alleles | Example Substragtes | Effects |
| 1A2 | 35 | Duloxetine, alosetron | ↓induction ↓expression |
| 2A6 | 50 | Nicotine, tegafur | ↑activity ↓activity |
| 2B6 | >50 | Efavirenz, | ↓activity |
| | | cyclophosphamide | |
| 2C8 | 16 | Replaginide | ↓activity |
| 2C9 | 35 | Warfarin, phenytoin | ↓activity |
| 2C19 | 25 | Omeprazole | ↑activity ↓activity |
| 2D6 | >100 | Desipramine | ↑metabolism |
| | | | ↓metabolism |
| 2E1 | 13 | Isoniazid | ↓expression |
| 3A4 | 40 | Eplerenone, simvastatin | Polymorphic expression |
| 3A5 | 20 | Tacrimolus | Polymorphic expression |

CYP: cytochrome P450 drug metabolizing enzymes

TABLE 4
Number of alleles with selected phase-II enzyme
polymorphisms and functional effects thereof

| | Number | | |
|---|---|---|---|
| | of | | Functional |
| Gene | Alleles | Example Substrates | Effects |
| TPMT | ≧20 | Thiopurines | ↓activity |
| COMT | ≧2 | Dobutamine, L-dopa | ↓activity |
| SULT1A1 | >15 | Acetaminophen, minoxidil | ↓activity |
| NAT2 | >50 | Isoniazid, amonoafide | ↓activity |
| UGT1A1 | >30 | Irinotecan | ↓express |
| UGT2B7 | >30 | Morphine, zidovudine | ↓activity |
| GSTM | >3 | Metabolites of polyaromatic | ↓expression |
| | | hydrocarbons | |
| GSTT | ≧1 | Halogenated hydrocarbons | ↓expression |

TPMT: thiopurine methyltransferase
COMT: catecho O-methyltransferase
SULT: sulfotransferase
NAT: N-acetyltransferase
UGT: uridine glucuronosyltransferase
GST: glutathone-S-transferase

TABLE 5
Number of alleles with selected drug transporter
polymorphisms and functional effects thereof

| | Number | | |
|---|---|---|---|
| | of | | Functional |
| Gene | Alleles | Example Substrates | Effects |
| ABCB1 | ≧100 | Digoxin, fexofenadine | Unclear |
| ABCC2 | ≧200 | Indinavir, cisplatin, drug | ↑activity |
| | | conjugates | ↑expression |
| ABCG2 | >40 | Doxorubicin, rosuvastatin | ↓expression |
| SLC01B1 | >20 | Pravastatin, rifampin | ↓affinity |
| SLC22A1 | | Metformin, desipramine | ↓affinity |
| (OCT1) | | | |

ABC: ATP binding cassette transporters
SLC: solute carrier transporters

As is apparent from the above Tables 3, 4 and 5, the present invention suggests that, if a metabolic enzyme or transporter as a xenobiotic delivered by a drug metabolizing enzyme (DME) or transporter has differences between individuals caused by genetic polymorphisms thereof, systematic utilization of the foregoing genetic profiling information in BA or BE test may improve the BA or BE test.

Hereinafter, the present invention will be more particularly described by the preferred examples. However, these are intended to illustrate the invention as preferred embodiments of the present invention and do not limit the scope of the present invention.

### Example 1

For each of 50 BE tests performed in the present invention, a CV_{w} and a CV_{b}, for Cₘₐₓ were calculated according to Equations 2 and 3, while 90% CI was calculated according to Equation 1 described above.⁸

CV_{b}, that is, a within-subject coefficient of variation may be defined as follows: ⁸

CV_{w}=\sqrt{exp(σ_{w}²)−1} Equation 2

CV_{b}, that is, a between-subject coefficient of variation may be defined as follows:⁸

$$C {\hat{V}}_{b} = \sqrt{exp ( \frac{{MS}_{between} - {MS}_{within}}{2} ) - 1} \qquad Equation 3$$

A natural log-transformed CV_{w} and a natural log-transformed CV_{b} were applied to a linear regression according to a SAS program (SAS 9.1.3, SAS Institute Inc., Cary, N.C., USA) to obtain the following results. FIG. 2 shows a relation between the CV_{w} and the CV_{b} as described above.

The REG Procedure
Model: MODEL1
Dependent Variable: WITHIN
Analysis of Variance

| | | Sum of | Mean | | |
|---|---|---|---|---|---|
| Source | DF | Squares | Square | F Value | Pr > F |
| Model | 1 | 5.67256 | 5.67256 | 43.99 | <.0001 |
| Error | 48 | 6.18904 | 0.12894 | | |
| Corrected Total | 49 | 11.86160 | | | |
| Root MSE | | 0.35908 | R-Square | 0.4782 | |
| Dependent Mean | | −1.33853 | Adj R-Sq | 0.4674 | |
| Coeff Var | | −26.82652 | | | |

Parameter Estimates

| | | Parameter | Standard | | |
|---|---|---|---|---|---|
| Variable | DF | Estimate | Error | t Value | Pr > \|t\| |
| Intercept | 1 | −0.49736 | 0.13661 | −3.64 | 0.0007 |
| BETW | 1 | 0.61820 | 0.09320 | 6.63 | <.0001 |

Number of Observations Read 50
Number of Observations Used 50

It was observed as a linear proportional relation between the natural log-transformed CV_{w} and the natural log-transformed CV_{b}, with a gradient of 0.618 (p<0.0001). As shown in FIG. 2, it was found that there is a proportional relation between the within-subject variability and the between-subject variability for Cₘₐₓ. In other words, for Cₘₐₓ as a PK parameter, a positive correlation between the between-subject variability and the within-subject variability according to Equation 4 was surprisingly disclosed in the present invention, thus demonstrating novelty and inventiveness of the present invention.

Equation 4:

LN(CVw)=0.618×LN(CVb)−0.4974 Regression Equation

From Equation 1, a size of the CI in the BE test is proportional to an extent of CV_{w}. As shown in FIG. 2, it was observed that the within-subject variability is proportional to the between-subject variability according to Equation 4.

Briefly, it may be expected that the within-subject variability is decreased as the between-subject variability is decreased.

Alternatively, for each of 50 BE tests performed in the present invention, a 90% CI for C. was calculated according to the SAS program, while a natural log-transformed 90% CI and a natural log-transformed CV_{w} were applied to a linear regression according to the SAS program to obtain the following results.

The REG Procedure
Model: MODEL1
Dependent Variable: CI
Analysis of Variance

| | | Sum of | Mean | | |
|---|---|---|---|---|---|
| Source | DF | Squares | Square | F Value | Pr > F |
| Model | 1 | 6.68625 | 6.68625 | 109.68 | <.0001 |
| Error | 48 | 2.92621 | 0.06096 | | |
| Corrected Total | 49 | 9.61247 | | | |
| Root MSE | | 0.24691 | R-Square | 0.6956 | |
| Dependent Mean | | −1.44644 | Adj R-Sq | 0.6892 | |
| Coeff Var | | −17.06993 | | | |

Parameter Estimates

| | | Parameter | Standard | | |
|---|---|---|---|---|---|
| Variable | DF | Estimate | Error | t Value | Pr > \|t\| |
| Intercept | 1 | −0.44148 | 0.10212 | −4.32 | <.0001 |
| CVw | 1 | 0.75079 | 0.07169 | 10.47 | <.0001 |

Number of Observations Read 50
Number of Observations Used 50

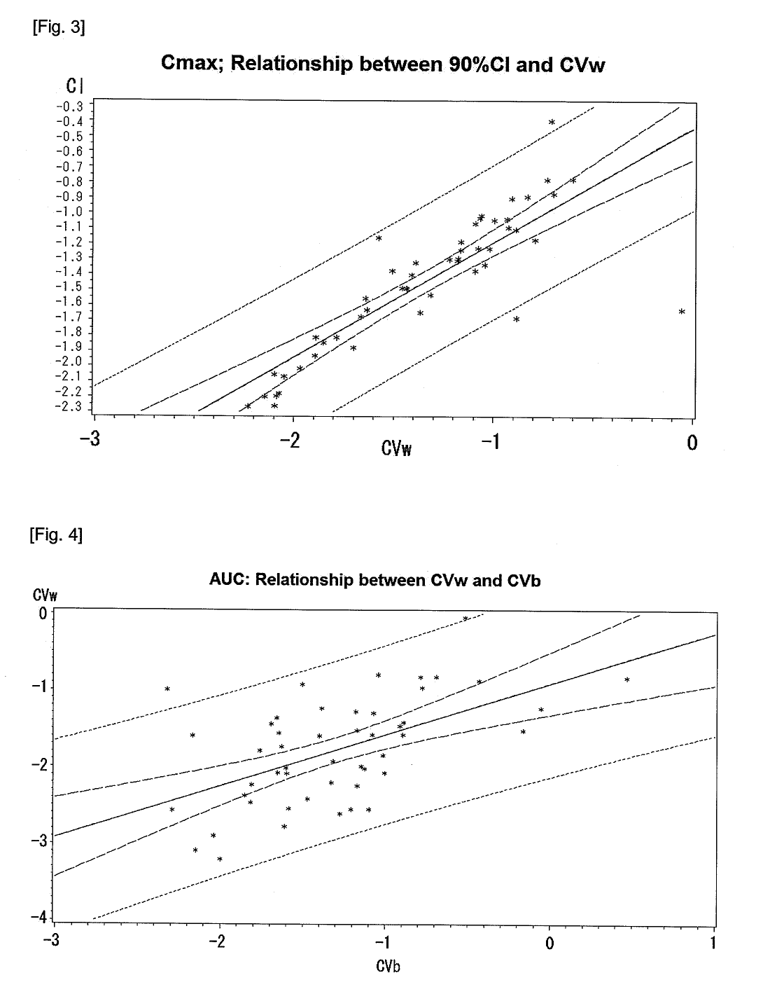

It was observed as a linear proportional relation between the natural log-transformed CI and the natural log-transformed CV_{w} with a gradient of 0.751 (p<0.0001). FIG. 3 shows the observed linear proportional relation. As shown in FIG. 3, it was observed as a positive correlation between an extent of the within-subject variability and the 90% CI according to the following Equation 5.

Equation 5:

LN(CI)=0.751×LN(CV_{W})−0.441 Regression Equation

If the between-subject variability is reduced according to FIGS. 2 and 3, the within-subject variability may be reduced which in turn enables a more narrowed CI range, thereby improving statistical power. Briefly, it may be expected to efficiently decrease a range of the CI without increasing the number of participants.

Meanwhile, among various causes of the between-subject variability, the most significant one may be a genetic polymorphism of a metabolic enzyme and/or transporter regarding in vivo activity of a drug.

Accordingly, the between-subject variability may be reduced by selecting individuals only who have the same or similar genetic polymorphisms of metabolic enzyme and/or transporter. Therefore, from the proportional relation between the between-subject variability and the within-subject variability first disclosed in the present invention, the within-subject variability may be reduced.

### Example 2

For each of 50 BE tests performed in the present invention, a CV_{w} and a CV_{b} for AUC were calculated according to Equations 2 and 3, while a 90% CI was calculated according to Equation 1.

A natural log-transformed CV_{w} and a natural log-transformed CV_{b} were applied to a linear regression according to a SAS program (SAS 9.1.3, SAS Institute Inc., Cary, N.C., USA) to obtain the following results. FIG. 4 shows a correlation between the CV_{w} and the CV_{b} for AUC as described above.

The REG Procedure
Model: MODEL1
Dependent Variable: CVw
Analysis of Variance

| | | Sum of | Mean | | |
|---|---|---|---|---|---|
| Source | DF | Squares | Square | F Value | Pr > F |
| Model | 1 | 7.15432 | 7.15432 | 21.90 | <.0001 |
| Error | 48 | 15.68115 | 0.32669 | | |
| Corrected Total | 49 | 22.83547 | | | |
| Root MSE | | 0.57157 | R-Square | 0.3133 | |
| Dependent Mean | | −1.78640 | Adj R-Sq | 0.2990 | |
| Coeff Var | | −31.99548 | | | |

Parameter Estimates

| | | Parameter | Standard | | |
|---|---|---|---|---|---|
| Variable | DF | Estimate | Error | t Value | Pr > \|t\| |
| Intercept | 1 | −0.91674 | 0.20266 | −4.52 | <.0001 |
| CVb | 1 | 0.66842 | 0.14283 | 4.68 | <.0001 |

Number of Observations Read 50
Number of Observations Used 50

It was observed as a linear proportional relation between the natural log-transformed CV_{w} and the natural log-transformed CV_{b}, according to Equation 6, wherein a gradient is 0.668 (p<0.0001) (see FIG. 4). That is, it was observed as a proportional relation between the within-subject variability and the between-subject variability.

For AUC as a PK parameter, a positive correlation between the between-subject variability and the within-subject variability according to Equation 6 was surprisingly found in the present invention, thus demonstrating novelty and inventiveness of the present invention.

Equation 6:

LN(CV_{w})=0.668×LN(CV_{b})−0.917 Regression Equation

From 50 BE tests performed in the present invention, a 90% CI for AUC was calculated and transformed into a natural log value. The natural log-transformed CI as well as a natural log-transformed CV_{w} were applied to a linear regression according to the SAS program, resulting in the following results.

The REG Procedure
Model: MODEL1
Dependent Variable: CI
Analysis of Variance

| | | Sum of | Mean | | |
|---|---|---|---|---|---|
| Source | DF | Squares | Square | F Value | Pr > F |
| Model | 1 | 15.21716 | 15.21716 | 326.35 | <.0001 |
| Error | 48 | 2.23819 | 0.04663 | | |
| Corrected Total | 49 | 17.45535 | | | |
| Root MSE | | 0.21594 | R-Square | 0.8718 | |
| Dependent Mean | | −1.89023 | Adj R-Sq | 0.8691 | |
| Coeff Var | | −11.42387 | | | |

Parameter Estimates

| | | Parameter | Standard | | |
|---|---|---|---|---|---|
| Variable | DF | Estimate | Error | t Value | Pr > \|t\| |
| Intercept | 1 | −0.43195 | 0.08631 | −5.00 | <.0001 |
| CVw | 1 | 0.81632 | 0.04519 | 18.07 | <.0001 |

Number of Observations Read 50
Number of Observations Used 50

It was observed as a linear proportional relation between a natural log-transformed 90% CI and a natural log-transformed CV_{w} with a gradient of 0.816 (p<0.0001). FIG. 5 shows a relation between the CI and the CV_{w} for AUC as described above. As shown in FIG. 5, it was observed as a positive correlation between an extent of the within-subject variability and the 90% CI according to Equation 7.

Equation 7:

LN(CI)=0.816×LN(CV_{W})−0.431 Regression Equation

Meanwhile, among various causes of the between-subject variability for AUC, the most significant one may be a genetic polymorphism of a metabolic enzyme and/or transporter regarding in vivo activity of a drug. Accordingly, the between-subject variability may be reduced by selecting individuals who only have the same or similar genetic polymorphisms of metabolic enzymes and/or transporters. Therefore, from the proportional relation between the between-subject variability and the within-subject variability first disclosed in the present invention, the within-subject variability may be reduced.

### Example 3

Risperidone (Janssen Korea) which is well known to be mostly metabolized by CYP2D6 as one of cytochrome metabolic enzymes¹⁰ and to have a known genetic polymorphism was orally administered to each of 17 healthy adult men in a dose of 3 mg, followed by periodic blood collection at 0.25, 0.5, 1, 2, 3, 4, 6, 8, 12, 24, 36 and 48 hours after the administration. Concentration of risperidone ingredient in blood plasma was quantified by means of validated LC-MS/MS method.

Quantification of risperidone by LC-MS/MS method is performed as follows:

After preparing 1 mg/mL of risperidone in 50% methanol as a free base, the solution was stored in a refrigerator. Plasma samples were prepared by dissolving this solution in a blank plasma stored in a freeze-drier and adjusting concentration of risperidone ingredient in the plasma samples up to 0.2, 0.5, 1, 5, 10, 30 and 40 ng/mL, respectively. 200 μL of each standard plasma sample was added with 50 μL of an internal standard material, for example, desipramine with concentration of 1 μg/mL. After vortexing the mixture, 25 μL of aqueous solution containing 2M sodium hydroxide and 1.2 mL of ethyl acetate were added thereto and further underwent the vortex agitation for 2 minutes. After centrifugation at 12,000 rpm for 5 minutes, 1 mL of supernatant was taken from the centrifuged solution, evaporated and dried for 30 minutes. Such pre-treated plasma sample was quantified under the following LC/MS/MS conditions. HPLC as a measurement apparatus and a triple quadruple mass spectrometer as a detector were used in this example, both of which are available from Waters Inc., in order to detect risperidone fraction (m/z 410.91>191.35) as well as desipramine as the internal standard material (m/z 267.09>208.22). In this case, MRM (multiple reaction monitoring) method was adopted. A column used in this example was Capcell Pak C₁₈UG 120V (5.0 μm, 2.0 mm×150.0 mm) equipped with Alltech Replacement prefilter having dimension of 4.0 mm×2.0 μm, while data processing equipment was a MassLynx integrator available from Waters Inc. Mobile phase of the column was a mixture solution that contained 10 mM ammonium formate buffer adjusted to pH 3.5 with formic acid/acetonitrile in volume ratio of 15/85 (v/v). The measurement was carried out at flow rate of the mobile phase of 0.25 mL/min.

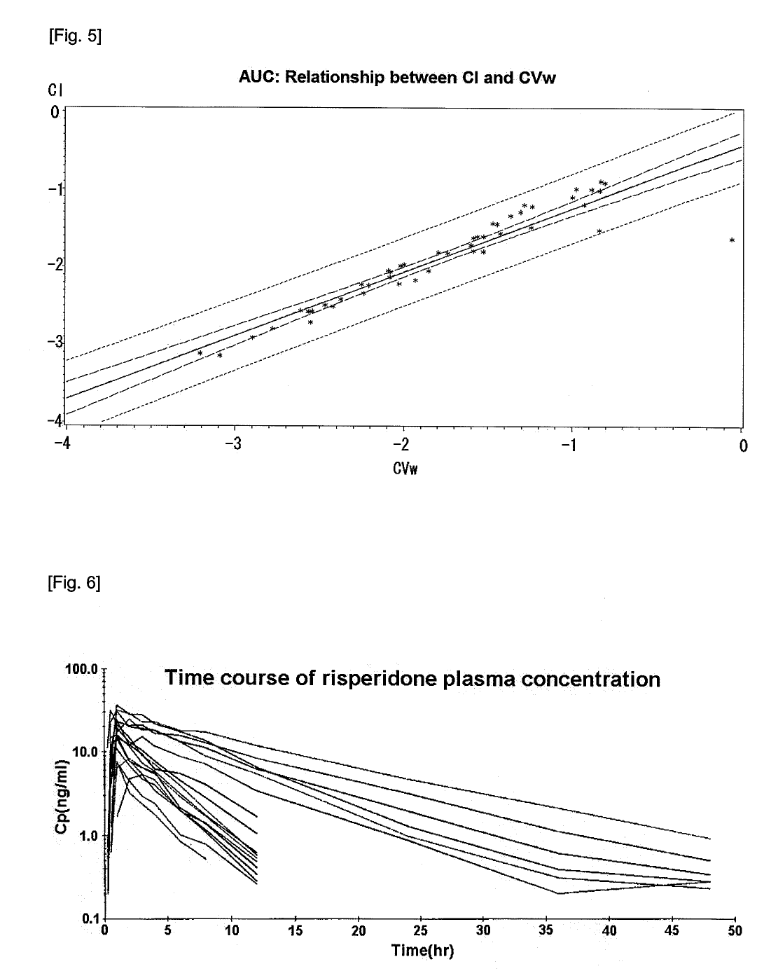

FIG. 6 shows concentration of risperidone in blood plasma for all of 17 participants. With the plasma samples described above, AUC_{LAST} (AUC until the final concentration is measured) was calculated from a curve of risperidone concentration vs. time by Trapezoidal Rule according to Equation 8 and Cₘₐₓ was calculated using experimental values.

$$Trapezoidal rule \text{:} AUC = \frac{1}{2} {\sum }_{i = 0}^{n - 1} ( {t}_{i + 1} - {t}_{i} ) ( {C}_{i + 1} + {C}_{i} ) \qquad Equation 8$$

wherein Cᵢ is concentration of drug in blood at measured time of tᵢ.

In parallel, participants as test subjects underwent gene study with written consent before the study. The gene study was performed as follows: DNA was extracted from blood cells and examined for allele of CYP2D6*2, CYP2D6*5 and CYP2D6*10 by means of RT-PCR method. Concentration of risperidone fraction in blood plasma is substantially not affected by genotype of CYP2D6*2 and/or CYP2D6*5. Whereas, it was observed that concentration of risperidone fraction in blood plasma is significantly varied depending on genotype of CYP2D6*10.

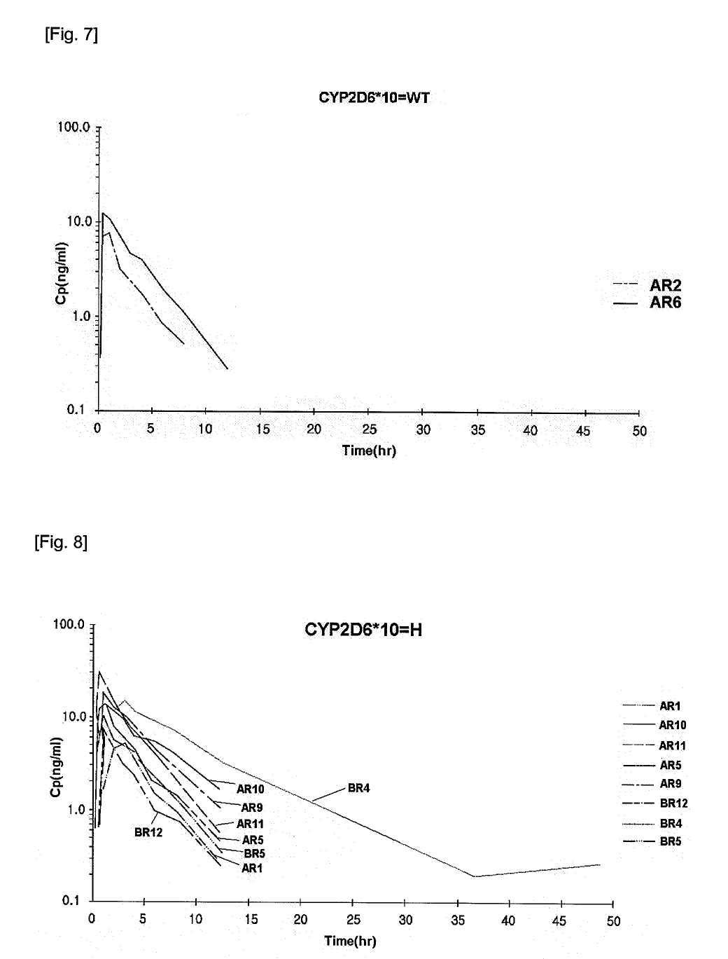

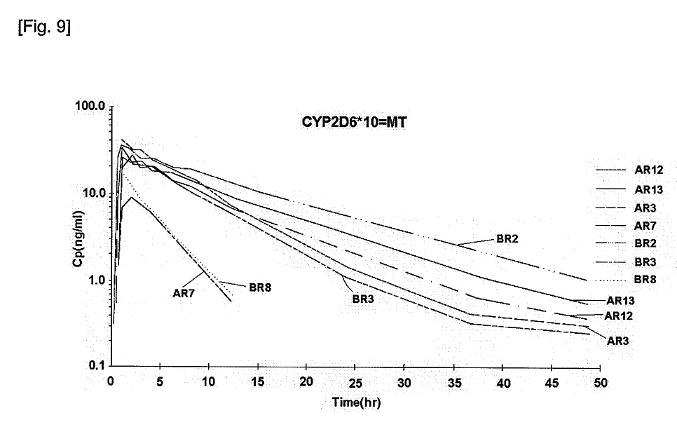

FIG. 7 shows concentration of risperidone fraction in blood plasma for some of test participants with wild type CYP2D6*10, FIG. 8 shows concentration of risperidone fraction in blood plasma for some of participants with heterozygous type CYP2D6*10, and FIG. 9 shows concentration of risperidone fraction in blood plasma for some of participants with mutant type CYP2D6*10.

Such calculated Cₘₐₓ was log-transformed to obtain mean value, standard deviation, variance, between-subject variability and/or CI, which are listed in Table 6 based on genotypes of CYP2D6 as the main metabolic enzyme of risperidone.

TABLE 6
Statistics of log-transformed Cₘₐₓ value

| | Without | With genetic profiling | | | |
|---|---|---|---|---|---|
| | genetic | Wild | Heterozygous | Mutant | |
| | profiling | Type (W) | Type (H) | Type (M) | W + H |
| N | 17 | 2 | 8 | 7 | 10 |
| Mean | 2.801 | 2.259 | 2.674 | 3.101 | 2.591 |
| CI (95%) | 2.50, 3.10 | −0.51, | 2.18, 3.17 | 2.63, 3.57 | 2.19, |
| | | 5.03 | | | 2.99 |
| Variance | 0.339 | 0.095 | 0.345 | 0.262 | 0.309 |
| STD | 0.582 | 0.308 | 0.587 | 0.512 | 0.556 |
| CV_{b} | 0.2078 | 0.1363 | 0.2195 | 0.1651 | 0.2146 |

CI: confidence interval (95%)
STD: standard deviation
CV_{b}: between-subject coefficient of variation

From the above Table 6, it can be seen that CV_{b} of Cₘₐₓ values of risperidone undergoing metabolism of CYP2D6 for all of participants without classification of genotypes is 20.8% while CV_{b}, for the participants having wild type CYP2D6 only is 13.6%, which is considerably lowered as compared to the former.

Since it was found that the between-subject variability is correlated with the within-subject variability according to Equation 4, applying CV_{b}, of Cₘₐₓ values of risperidone to Equation 4 to calculate CV_{w} may provide results shown in the following Table 7.

TABLE 7
Calculation of number of test examples for Cₘₐₓ of risperidone

| | Pooled; Without | |
|---|---|---|
| | Genotyping | Wild type only |
| CV_{b} | 0.2078 | 0.1363 |
| CV_{w} | 0.2357 | 0.1775 |
| Test | 0.9, 0.95, 1.0, | 0.9, 0.95, 1.0, |
| drug/control | 1.05, 1.1 | 1.05, 1.1 |
| drug ratio | | |
| Power | 90 | 90 |
| N | 70, 34, 26, 34, 60 | 42, 20, 16, 20, 36 |

As a program for calculation of statistical power of BE test and sample sizes, nQuery Advisor® (Ver.6.0, Statistical solutions, MA, USA) using an algorithm proposed by Diletti et al⁸. was used in order to estimate a statistical power and a sample size in BE test. The statistical power may be estimated using within-subject CV.

Using CV_{w} values obtained from Equation 4, the number of samples required for obtaining a statistical power of 90% (1−β) in BE test was calculated for a T/R ratio of 0.9 to 1.1 between test formulations and control formulations by means of nQuery Advisor® (Ver. 6.0, Statistical Solutions, MA, USA). The results are shown in the above Table 7.

For risperidone, in a case whereby the genetic profiling information was not applied, 26 participants were required in order to obtain a statistical power of 90% when the ratio of Cₘₐₓ mean values of test formulation to control formulation is 1.00. However, when the experiment was performed for participants with only wild type CYP2D6*10, the required number thereof was 16 (see Table 7).

The above difference in number of participants is significant in view of management of the entire BE test or economical aspect from the standpoint of a manufacturer. As shown in FIG. 7, the wild type participants have considerably low concentrations of risperidone in blood plasma, as compared to the participants with heterozygous type (FIG. 8) or mutant type (FIG. 9), and it can be seen that the concentration of risperidone in blood plasma is rapidly reduced.

Although the concentration of risperidone in blood plasma of the participants with only wild type was reduced below a measurable level 24 hours after dosing, the participants with mutant type still exhibited the concentration of risperidone in blood plasma capable of being measured even 48 hours after dosing. Accordingly, in the case where persons having only wild type are selected as participants, a blood taking time may be significantly shortened. Moreover, since the concentration of a drug in blood plasma is relatively low, an expression rate of side effects due to the drug may be decreased.

Briefly, participants with wild type metabolic enzymes and transporters undergo the BA/BE test and side effects possibly occurring during the test are decreased, thereby preventing problems in management of participants and intermediate dropouts.

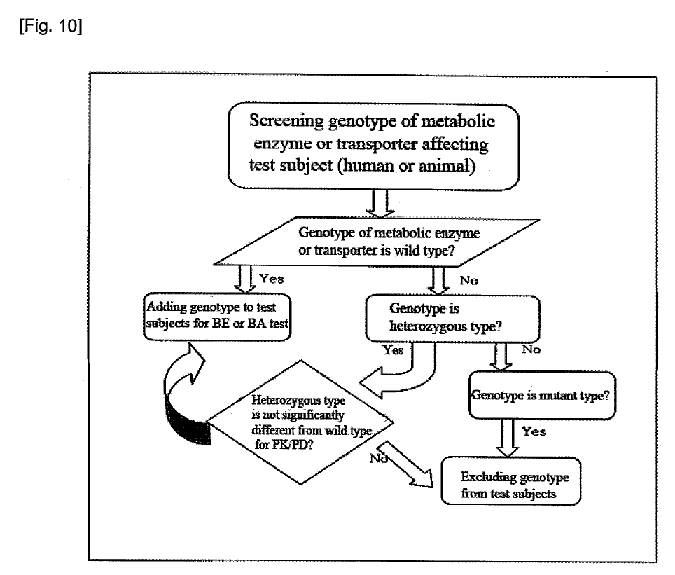

In the foregoing Examples 1 to 3, examining polymorphisms of metabolic enzyme or transporter that has influence on PK/PD, test subjects (human or animal) were selected and the BA/BE test was carried out for the selected test subjects (FIG. 10). As a result, when the same number of test examples was applied, it was observed that a rate of manufacturer risk is decreased (an increase in statistical power) and a 90% CI range is narrowed, thus enhancing a success rate of the BE test.

In other words, significantly decreasing the number of test subjects required for obtaining a certain statistical power for BA/BE test may remarkably improve management of the test and may reduce test costs.

### Example 4

Further, mean value, variance, standard deviation and/or CV_{b} value for AUC_{LAST} for each of genotypes which were obtained from test results for risperidone according to the present invention are shown in Table 8.

TABLE 8
Statistics of log-transformed AUC_{LAST} value

| | | With genetic profiling | | | |
|---|---|---|---|---|---|
| | | | | | Wild and |
| | Without genetic | Wild | Heterozygous | Mutant | heterozygous |
| | profiling | type | type | type | pooled |
| N | 17 | 2 | 8 | 7 | 10 |
| Mean | 4.322 | 3.264 | 3.939 | 5.061 | 3.804 |
| CI (95%) | 3.828, 4.816 | −1.14, 7.67 | 3.41, 4.47 | 4.27, 5.86 | 3.34, 4.27 |
| Variance | 0.923 | 0.240 | 0.407 | 0.739 | 0.424 |
| STD | 0.961 | 0.490 | 0.683 | 0.859 | 0.652 |
| CV_{b} | 0.2223 | 0.1501 | 0.1734 | 0.1697 | 0.1714 |

CI: confidence interval
STD: standard deviation
CV_{b}: between-subject coefficient of variation

From the above Table 8, it can be seen that CV_{b} of AUC values of risperidone undergoing metabolism of CYP2D6 for all of participants without classification of genotypes is 22.2% while CV_{c}, for the participants having wild type CYP2D6 only is 15%, which is considerably lowered as compared to the former.

Since it was found that the between-subject variability is correlated with the within-subject variability according to Equation 6, applying CV_{b} of AUC values of risperidone to Equation 6 for calculation of CV_{w} may provide the results shown in the following Table 9.

TABLE 9
Calculation of number of test examples for AUC of risperidone

| | Pooled; Without | |
|---|---|---|
| | genotyping | Wild type only |
| CV_{b} | 0.2223 | 0.1501 |
| CV_{w} | 0.1464 | 0.1126 |
| Test | 0.9, 0.95, 1.0, | 0.9, 0.95, 1.0, |
| drug/control | 1.05, 1.1 | 1.05, 1.1 |
| drug ratio | | |
| Power | 90 | 90 |
| N | 70, 34, 26, 34, 60 | 42, 20, 16, 20, 36 |

As a program for calculation of statistical power of BE test and sample sizes, nQuery Advisor® (Ver.6.0, Statistical solutions, MA, USA) using an algorithm proposed by Diletti et al.⁸ was used in order to estimate a statistical power and a sample size in BE test. The statistical power may be estimated using CV_{w}.

Using CV_{w} values obtained from Equation 6, the number of samples required for obtaining a statistical power of 90% (1−β) in BE test was calculated for a T/R ratio of 0.9 to 1.1 between test formulations and control formulations by means of nQuery Advisor® (Ver. 6.0, Statistical Solutions, MA, USA). The results are shown in the below Table 10.

TABLE 10
Calculation of number of test examples for AUC of risperidone

| | Pooled; Without | |
|---|---|---|
| | genotyping | Wild type only |
| CV_{b} | 0.2223 | 0.1501 |
| CV_{w} | 0.1464 | 0.1126 |
| Test | 0.9, 0.95, 1.0, | 0.9, 0.95, 1.0, |
| drug/control | 1.05, 1.1 | 1.05, 1.1 |
| drug ratio | | |
| Power | 90 | 90 |
| N | 28, 16, 12, 14, 24 | 18, 10, 8, 10, 16 |

For risperidone as shown in Table 10, in a case whereby the genetic profiling information was not applied, test subjects were required in order to obtain a statistical power of 90% when the ratio of AUC mean values of test formulation to control formulation is 1.00. However, when the experiment was performed for participants with only wild type CYP2D6*10, the required number thereof was 8.

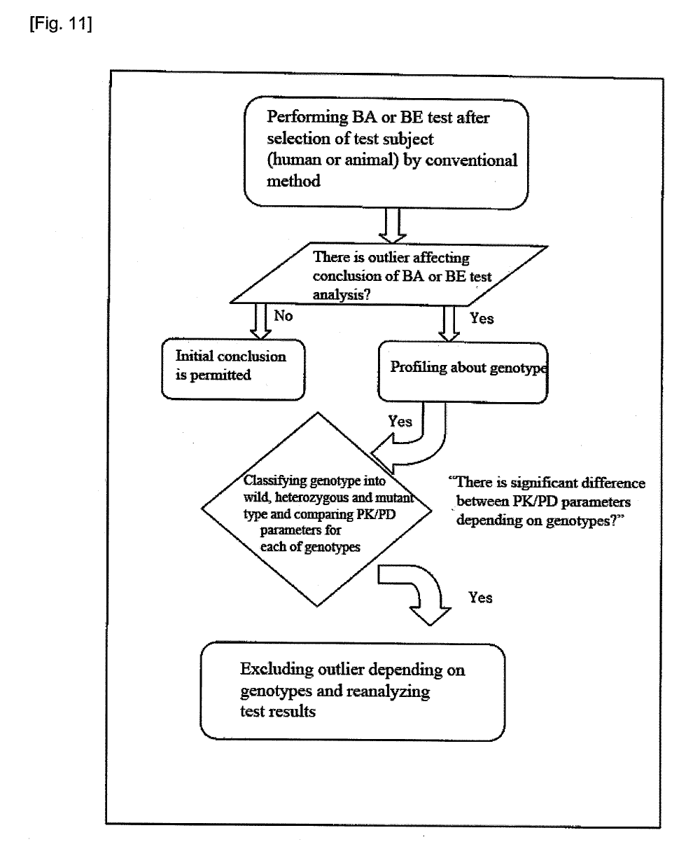

Consequently, as is apparent from the foregoing Examples 3 and 4, it is obvious that utilization of genetic profiling information has beneficial features and advantages in designing and performing BA/BE test. For BA/BE test, the genetic profiling information can be applied to test analysis, for example, before the test and after the test, in order to exclude a part of data for test subjects from statistical determination of the test or in order to include the data in the statistical determination (see FIG. 11). Alternatively, when calculating the number of test examples prior to the test, the genetic profiling information may also be utilized to determine a difference in the calculated number of test examples whereby the genetic profiling information is utilized or not.

For a drug having a very long half-life of more than 5 days,¹¹ the BE test is difficult to proceed by a crossover study. In this regard, a parallel study design is preferably employed. However, such parallel study design demands a great number of subjects since the between-subject variability is large in this study. However, if the between-subject variability is decreased by genetic profiling, a success rate of BA/BE test for a xenobiotic having a considerably long half-life may be enhanced using a parallel study design. Examples of drugs having long half-lives are listed in Table 11.

TABLE 11
Some drugs with long half-lives: lasting of blood sampling
and washout needed in a single dose, two-period, two-sequence,
two-formulation crossover bioequivalence study ¹¹

| | | Blood sampling | Wash-out |
|---|---|---|---|
| Drug | t_{1/2}(days) | Period(weeks) | Period(weeks) |
| Amiodarone | 50 | 21 | ≧50 |
| Digitoxin | 7 | 3 | 8 |
| Chloroquine | 41 | 17 | ≧45 |
| Tamoxifen and | 8/11 | 5 | ≧10 |
| demethyltamoxifen | | | |

### REFERENCE

1. US Food and Drug Administration, Guidance for Industry: bioavailability and bioequivalence studies for orally administered drug products-general considerations, Center for Drug Evaluation and Research. Rockville, Md., 2003.
2. US Food and Drug Administration, Statistical approaches to establishing bioequivalence. Guidance for industry. Rockville, Md., 2001.
3. EMEA/CPMP/EWP/1401/98. Note for guidance on the investigation of bioavailability and bioequivalence. EMEA, London, 2001.
4. Bioequivalence test guideline, Korea Food and Drug Administration (KFDA) published No. 2008-22.
5. C. E. Diliberti. Why bioequivalence of highly variable drugs is an issue. Advisory Committee for Pharmaceutical Sciences Meeting Transcript, 2004. http://www.fda.gov/ohrms/dockets/ac/04/transcripts/403 4T2.pdf.
6. K. K. Midha, M. J. Rawson and J. W. Hubbard. The bioequivalence of highly variable drugs and drug products. Int. J. Clin. Pharmacol. Ther. 43, 485-498, 2005.
7. B. M. Davit et al. Highly variable drugs: observations from bioequivalence data submitted to the FDA for new generic drug applications. The AAPS J. 10, 148-156, 2008.
8. D. Hauschke, et al. Presentation of the intrasubject coefficient of variation for sample size planning in bioequivalence studies. Int. J. Clin. Pharmacol. Ther. Toxicol. 32, 376-378, 1994.
9. J. A. Williams et al. PhRMA White paper on ADME pharmacogenomics. J. Clin. Pharmacol. 48, 849-889, 2008.
10. Norio Yasui-Furukori et al. Effects of CYP2D6 genotypes on plasma concentrations of risperidone and enantiomers of 9-hydroxyrisperidone in Japanese patients with schizophrenia. J. Clin. Pharmacol. 2003: 43, 122-127.
11. A. Marzo Open questions on bioequivalence: some problems and some solutions. Pharmacol. Res. 40, 357-368, 1999.

As described above, the present invention may embody various improvements in performance of BA or BE test, wherein test subjects are selected in consideration of genetic characteristics of metabolic enzymes or transporters that have influence on PK and/or PD for xenobiotics in order to reduce the between-subject variability and the within-subject variability, thereby enhancing statistical power of the BA/BE test while more obviously determining differences between formulations which in turn raises a success rate of the test. According to the inventive test method, it may be possible to decrease the number of test examples (number of test participants or animals) so as to reduce entire costs and time taken in BA/BE test and, in addition, to reduce incidence of expressing side effects or harmful effects of the xenobiotics to the test subject, thereby embodying improvements in view of human rights.

Moreover, genetic profiling information of the test subject may be utilized for analysis of BA/BE test results for xenobiotics after the test.

While the present invention has been described with reference to the preferred examples, it will be understood by those skilled in the art that various modifications and variations may be made therein without departing from the scope of the present invention as defined by the appended claims.

1-17. (canceled)

18. A test method for xenobiotics using a fact that there is a positive proportional correlation between a within-subject variability and a between-subject variability for pharmacokinetics (PK) parameters, comprising:
analyzing genomic information of metabolic enzymes or transporters that have influence on pharmacokinetics (PK) or pharmacodynamics (PD) for xenobiotics, so as to reduce the within-subject variability and the between-subject variability; and
selecting test subjects who have the same or similar genetic polymorphisms based on analyzed results, and then, carrying out a bioavailability (BA) test or a bioequivalence (BE) test of xenobiotics.

19. The test method according to claim 18, further comprising: analyzing a result of the BE test or the BA test based on the genomic information of the metabolic enzymes or transporters.

20. The test method according to claim 18, wherein the PK parameters comprise area under the blood concentration verse time curve (AUC) and the peak blood concentration of drug (Cₘₐₓ).

21. The test method according to claim 18, wherein the xenobiotic has a biological half-life of more than 5 days.

22. The test method according to claim 19, wherein the analysis of the result from the BA test or the BE test is conducted by at least one selected from a group consisting of a cross-over study, a parallel study and a repeated study.

23. The test method according to claim 18, wherein the genetic polymorphism is wild type, heterozygous type or mutant type.

24. The test method according to claim 23, wherein a specific test subject having a wild type genetic polymorphism is included in or excluded from the test subjects.

25. The test method according to claim 23, wherein a specific test subject having a heterozygous type genetic polymorphism is included in or excluded from the test subjects.

26. The test method according to claim 23, wherein a specific test subject having a mutant type genetic polymorphism is included in or excluded from the test subjects.

27. The test method according to claim 18, wherein after analyzing the genomic information of metabolic enzymes or transporters, the analyzed result is used to calculate number of test subjects required for the BA test or the BE test for the xenobiotics.

28. The test method according to claim 18, wherein the test is carried out in consideration of genetic pholymorphism of metabolic enzyme or transporter that has influence on pharmacokinetics (PK) or pharmacodynamics (PD) for xenobiotics in a test subject, so as to reduce side effects possibly observed in the test subject.

29. The test method according to claim 18, wherein a test subject with a wild type genetic polymorphism of metabolic enzyme or transporter that has influence on pharmacokinetics (PK) or pharmacodynamics (PD) for xenobiotics is selected and the test is carried out for the selected test subject, so as to shorten test time.

30. The test method according to claim 19, further comprising:
carrying out profiling about genetic polymorphisms of test subjects, if there is an outlier affecting a conclusion of analysis for BA or BE test;
classifying the genetic polymorphisms into wild type, heterozygous type and/or mutant type and comparing pharmacokinetics (PK) or pharmacodynamics (PD) parameters for each of the genetic polymorphisms; and,
if a significant difference between PK parameters or PD parameters dependent on the genetic polymorphism is recognized, analyzing the result after removing the outlier based on the genetic polymorphism.
